(19) United States
(12) Patent Application Publication
(10) Pub. No.: US 2009/0014351 A1
(43) Pub. Date: Jan. 15, 2009

(54) Adult incontinence kit

(75) Inventor: Charles B. Walker, Roanoke, VA (US)

Correspondence Address: BACON & THOMAS, PLLC, 625 SLATERS LANE, FOURTH FLOOR, ALEXANDRIA, VA, 22314-1176, US

(73) Assignee: AIMCO, LLC, Roanoke, VA (US)

(21) Appl. No.: 12/216,847

(22) Filed: Jul. 11, 2008

Related U.S. Application Data
(60) Provisional application No. 60/929,823, filed on Jul. 13, 2007.

(51) Int. Cl. A61F 13/551

(52) U.S. Cl. 206/570; 604/385.13

(57) ABSTRACT

A compact, convenient and portable prepackaged kit for use in aiding individuals suffering from adult incontinence and those who care for them is disclosed. The kit is useful both for providing all of the materials needed to tend to an individual suffering from adult incontinence in one convenient package and for providing a quick and convenient means by which to dispose of the materials after they have been used.

### CROSS-REFERENCE TO RELATED APPLICATIONS

This application claims the benefit of U.S. Provisional Application No. 60/929,823, filed Jul. 13, 2007, the whole of which is incorporated herein by reference.

### TECHNICAL FIELD

The invention described herein relates to an adult incontinence kit.

### BACKGROUND OF THE INVENTION

Incontinence refers to the lack of voluntary control of excretory functions. Fecal incontinence is the inability to control one's bowels. Urinary incontinence is the involuntary excretion of urine from one's body.

Adult incontinency is a growing challenge in long term care facilities across the nation, as up to 90% of residents in long term care facilities suffer from some degree of incontinency. Besides issues of odor and loss of dignity, adult incontinence may also result in wound and wound healing complications.

Adults suffering from incontinence often manage their bladder control problems through the use of disposable and absorbent underwear. However, in the case of a typical resident in a long term care residence, the disposable and absorbent underwear will need to be changed, normally with the aid of a nursing assistant, three or more times a day. This process may include cleaning, preventing and addressing wounds, sterilizing, applying protective ointment and disposing of used products in a sanitary manner.

Accordingly, it is necessary for a nursing assistant to have several products associated with adult incontinence at their immediate disposal when attending to a resident suffering from adult incontinence. In the past, this has required the nursing assistant to carry each item separately into the residents' rooms or has required the assistant to stack all of the different products on a movable cart and push the cart into the residents' rooms.

Carrying the items in to the residents' rooms separately is burdensome for assistants, especially in the instance when an assistant forgets to bring one of the essential items needed for tending to the resident. Carrying multiple items all at once can also be very cumbersome for an assistant. Further, because the resident may be embarrassed during this process, the failure of the assistant to have one of the items tends to delay the process and cause further loss of dignity to the resident.

In the case where a movable cart is used, the cart is often large and difficult to maneuver. Using a cart also does not remedy the issue of when one of the several items needed for treating a patient suffering from adult incontinency runs out before the others.

It is also true that neither carrying each item in separately nor stacking all of the items on a cart provides a suitable means for monitoring inventory of adult incontinence products or how many times a particular patient has been tended to during a specified period of time.

The present invention was designed with all of these problems in mind. A first object of the present invention is to provide a product that makes tending to adults suffering from incontinence more convenient for the caregiver and more time-efficient by including a disposable adult brief, a pair of disposable gloves, a disposable cleansing cloth and a pack of skin protection ointment in one convenient disposable, re-sealable impermeable bag.

Another object of the present invention is to aid nursing assistants and supervisors of long-term care facilities in monitoring inventory of adult incontinence products as well as how many times a resident in a long-term care facility suffering from adult incontinence has been tended to. This objective may be accomplished by setting out a certain number of adult incontinence kits as described herein per day per patient, such that if one or more adult incontinence kits assigned to a patient on a given day is not used at the end of a shift or the day, the nursing assistant or supervisor is put on notice to investigate and make necessary and timely corrections.

Other advantages and novel features of the present invention will become apparent in the following detailed description of the invention.

### SUMMARY OF THE INVENTION

The present invention relates generally to an adult incontinence kit. More particularly, the present invention relates to a compact, convenient and portable prepackaged kit for use in aiding individuals suffering from adult incontinence and those who care for them. The kit is useful both for providing all of the materials needed to tend to an individual suffering from adult incontinence in one convenient package and for providing a quick and convenient means by which to dispose of the materials after they have been used. The kit may also aid in inventory control and patient maintenance in long-term care facilities.

The first embodiment of the invention described herein is directed to an adult incontinence kit comprising a disposable, re-sealable impermeable bag; at least one disposable adult brief; at least one pair of disposable gloves; at least one disposable cleansing cloth; and at least one package of skin care ointment; wherein the at least one disposable adult brief, at least one pair of disposable gloves, at least one disposable cleansing cloth and at least one package of skin care ointment are housed in the disposable, re-sealable impermeable bag prior to use and wherein the disposable, re-sealable impermeable bag is capable of being used for collecting, sealing and disposing of the at least one disposable adult brief, the at least one pair of disposable gloves, the at least one disposable cleansing cloth and the at least one foil pack of skin care ointment after some or all of the items have been used.

The second embodiment of the invention described herein is directed to a method of providing for the incontinency needs of an individual comprising the steps of: providing a disposable, sealed and re-sealable impermeable bag housing at least one disposable adult brief, at least one pair of disposable gloves, at least one disposable cleansing cloth and at least one package of skin care ointment; storing the disposable, sealed and re-sealable impermeable bag in close proximity to an individual suffering from adult incontinence; opening the disposable, sealed and re-sealable impermeable bag and removing the at least one disposable adult brief, at least on pair of disposable gloves, at least one disposable cleansing cloth and at least on package of skin care ointment from the disposable, sealed and re-sealable impermeable bag; putting on the at least one pair of disposable gloves; optionally removing a soiled disposable adult brief from the individual suffering from adult incontinence; using the at least one cleansing cloth to remove soil from the individual suffering from adult incontinence; applying the skin care ointment contained in the package of skin care ointment to the where soil was removed from the individual suffering from adult incontinence; putting the at least one disposable adult brief on the individual suffering from adult incontinence; placing the used pair of disposable gloves, the used cleansing cloth, the used package of skin care ointment and optionally the used disposable adult brief removed from the individual suffering from adult incontinency in the disposable, re-sealable impermeable bag and resealing the disposable, re-sealable impermeable bag; and transporting the disposable, re-sealable impermeable bag to a disposal receptacle and depositing the disposable, re-sealable impermeable bag therein.

### BRIEF DESCRIPTION OF THE DRAWING

FIG. 1 is a front view of an adult incontinence kit in accordance with the present invention.

### DETAILED DESCRIPTION OF INVENTION

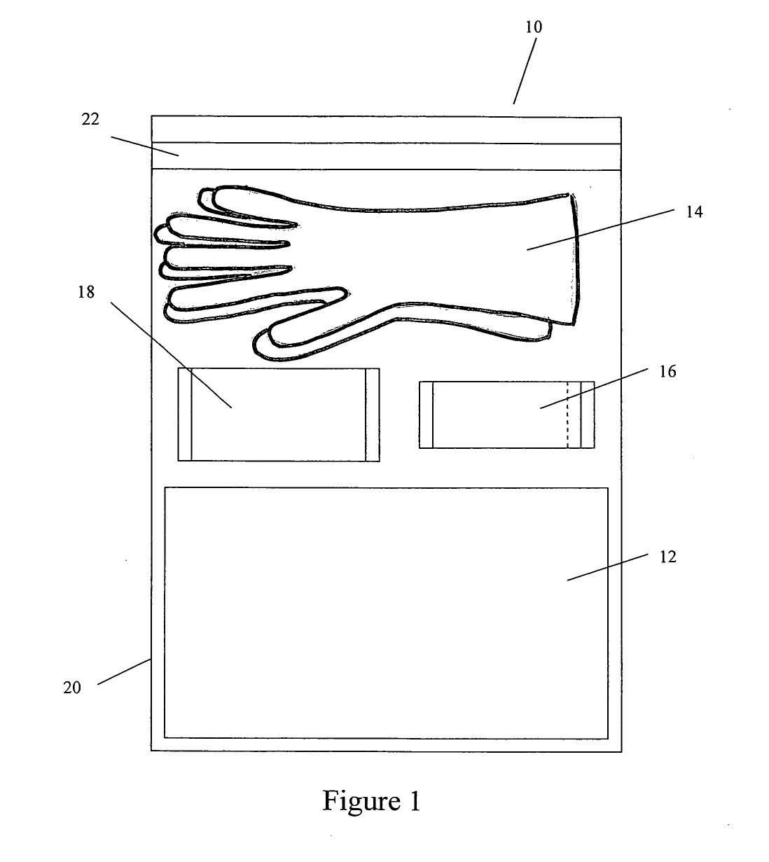

In the first embodiment of the invention described herein, the adult incontinence kit 10 generally includes a disposable adult brief 12, a pair of disposable gloves 14, a pack of skin ointment 16 having softening, soothing and preferably analgesic effects and a disposable cleansing cloth 18, all of which are packaged in a disposable, impermeable re-sealable bag 20 that is preferably large enough to hold each of the previously listed items as shown in FIG. 1. The disposable, impermeable re-sealable bag 20 is designed to compactly hold the disposable adult brief 12, a pair of disposable gloves 14, pack of skin care ointment 16 and disposable cleansing cloth 18 before use and also to hold the used gloves, used cleansing cloth and used pack of skin care ointment, along with a used adult brief. Once the used items have been placed in the disposable, impermeable re-sealable bag 20, the bag can be sealed by means of a seal 22 and disposed of safely and odor free.

The disposable, impermeable re-sealable bag suitable for use in the adult incontinence kit described herein may be any type of bag that is disposable, water-tight and re-sealable. If the situation requires, the disposable, impermeable re-sealable bag may be suitable for holding biohazardous waste. By “biohazard” it is meant a biological agent, such as a virus or a condition that constitutes a threat to humans. By “biohazard bag” it is meant a bag capable of safely containing and preventing the spread of a material that may be or contains a biohazard. By “disposable” it is meant capable of being discarded immediately after being used or used up, either through normal trash receptacles or specific biohazardous trash receptacles, depending on governmental regulation and health facility protocol.

The bag may typically be made of polyurethane or polypropylene, but if necessary may be of made of any material suitable for holding human waste. The thickness of the bag is preferably at least 1.5 mm thick, but may be any thickness that is suitable for holding human waste. The bag may also optionally include a biohazard label to indicate that the contents of the bag are bio-hazardous. The bag is also re-sealable, so that once the original contents are removed from the bag and replaced with used items, such as a used adult brief, used pair of disposable gloves, used disposable cleansing cloth and used pack of skin ointment, the bag can be resealed to ensure the used contents are securely contained within the bag. Any means for resealing the bag may be used, for example, an adhesive strip or zipper.

In one aspect of the first embodiment, the disposable, impermeable re-sealable bag is opaque, so that the contents are not visible from outside the bag. In this manner, the used items placed in the bag are not perceptible from outside of the bag. The disposable, impermeable, re-sealable bag may also have other print on it, such as the contents of the bag and other information pertaining to the products contained therein, e.g., the size of the adult brief.

The disposable adult brief suitable for use in the adult incontinence kit described herein may be any type of full-fitting disposable and absorbent adult brief. The material of the adult brief may be any suitable material that is both absorbent and disposable. Typical disposable adult briefs are often made of an outer layer of waterproof polyethylene, an inside layer of wood pulp and synthetic polyacrylate (absorbent crystala) and a water-repelling liner. The disposable adult brief may be of any size suitable for fitting an adult, and the bag which the items are placed in may be marked to indicate the size of disposable adult brief contained therein to assist in providing the appropriate adult incontinence kit to different sized individuals. The adult brief may also include repositionable straps to allow for use by adults with many different sizes and to insure a better fit. The disposable adult brief may also vary in its absorbency, such as medium absorbency or super absorbency, to cater to the degree of incontinency that the individual suffers from. The absorbent areas of the disposable adult brief may also be designed to better suit a male or female, or may also be of a unisex design. The disposable brief may also have soft side leg elastic and a fluted waistband to provide additional leakage control. The disposable adult brief may also optionally have an added fragrance to mask smell.

In another aspect of the first embodiment, the gloves are either latex or non-latex gloves. The gloves are preferably a high-quality, medical grade glove. The gloves may be ambidextrous, powdered or non-powdered, textured or untextured and may be of any size, but preferably a size large enough to fit many different size hands. The material of the latex gloves is preferably 100% latex, but also may comprise a blend of materials in addition to latex. The gloves should be chemically resistant to, for example, acids, alcohols, ketones and alkalies. The adult incontinence kit may include several pairs of disposable gloves, and preferably includes at least one pair of disposable gloves.

The disposable cleansing cloth included in the adult incontinence kit described herein may be any type of cleansing cloth commonly available on the market. The material of the cleansing cloth may be, for example, spun lace material. The disposable cleaning cloth is preferably a prepackaged individual-use cleansing cloth that is disposable after use. The cleansing cloth may also be flushable. The cleansing cloth may be pre-moistened, hypoallergenic and may optionally contain additives such as aloe, vitamin E, and fragrance to mask smell. The adult incontinence kit may contain multiple disposable cleansing cloths, and preferably contains at least one disposable cleaning cloth.

The adult incontinence kit may also include a pack of skin care ointment for application to the residents' skin during the process of changing the disposable adult brief. The skin care ointment should be capable of leaving the skin soft and dry and also protect the skin from breakdown associated with incontinence. The pack of skin ointment is preferably a single use foil pack of skin ointment wherein the skin ointment is of the type used to prevent rashes and skin breakdown. The pack of skin ointment should be easy to open, and may preferably have a means for quick opening, such as a perforation.

In a second embodiment of the invention described herein, a method of providing for the incontinency needs of an individual is disclosed. The method preferably comprises the steps of a) providing a disposable, sealed and re-sealable impermeable bag housing at least one disposable adult brief, at least on pair of disposable gloves, at least one disposable cleansing cloth and at least on package of skin care ointment; b) storing the disposable, sealed and re-sealable impermeable bag in close proximity to an individual suffering from adult incontinence; c) opening the disposable, sealed and re-sealable impermeable bag and removing the at least one disposable adult brief, at least one pair of disposable gloves, at least one disposable cleansing cloth and at least one package of skin care ointment from the disposable, sealed and re-sealable impermeable bag; d) putting on the at least one pair of disposable gloves; e) optionally removing a soiled disposable adult brief from the individual suffering from adult incontinence; f) using the at least one cleansing cloth to remove soil from the individual suffering from adult incontinence; g) applying the skin care ointment contained in the package of skin care ointment to the area where soil was removed from the individual suffering from adult incontinence; h) putting the at least one disposable adult brief on the individual suffering from adult incontinence; i) placing the used pair of disposable gloves, the used cleansing cloth, the used package of skin care ointment and optionally the used disposable adult brief removed from the individual suffering from adult incontinency from step e) in the disposable, re-sealable impermeable bag; j) resealing the disposable, re-sealable impermeable bag; and k) transporting the disposable, re-sealable impermeable bag to a disposal receptacle and depositing the disposable, re-sealable impermeable bag therein.

With respect to steps a), d), f), g), h) and i), these steps are self-explanatory and do not require further explanation.

With respect to step b), close proximity to the individual suffering from adult incontinency preferably means within the room of the adult suffering from adult incontinency, and more preferably, within a 2-3 foot radius of the individual. Storing can mean placing in a closet, in a drawer or any other area where the adult incontinence is easily accessible when needed but not in the way when not needed.

With respect to step c), opening the disposable, re-sealable impermeable bag preferably means opening the bag such that the re-sealable means is not destroyed or affected.

With respect to step e), optionally removing a soiled disposable adult brief from the individual suffering from adult incontinence will be necessary when the adult is already managing their adult incontinency through the use of disposable and absorbent adult briefs.

With respect to step j), resealing means using whichever means for resealing the disposable impermeable bag is provided in order to contain the used items within the disposable impermeable bag. Means for resealing the bag are not limited and may include, for example, a zipper or adhesive strip as described in the first embodiment of the present invention.

With respect to step k) transporting the disposable impermeable bag to a disposal receptacle may be by any means suitable for moving the disposable impermeable bag to an area of disposal, including but not limited to carrying by hand or placing in a temporary disposal bin. The disposal receptacle may be any receptacle capable of containing the disposable, impermeable bag which is used for storage prior to a waste management provider collecting the disposable, impermeable bags contained within the disposal receptacle and transporting the disposable, impermeable bags to, for example, a land fill or incineration plant. As discussed in the first embodiment, the disposable bag may also be a biohazard bag, and in such case, the disposal would be pursuant to regulations governing the disposal thereof.

The adult incontinence kit described herein may also be used beneficially to track the number of times that a resident in a long term care facility has been tended to by a nurse's assistant. For example, if past history of the resident has shown that that the resident requires his or her adult briefs be changed three times a day, then a supervisor may assign three adult incontinence kits to the patient per day. If one or more of the adult incontinence kits are remaining at the end of the day, the supervisor is put on notice to investigate and make necessary and timely corrections. In this way, use of the adult incontinence kits described herein may be used in a manner to assure that nursing assistants change the residents the appropriate number of times per day based on the past history of the residents needs related to adult incontinency.

### VARIATIONS

The foregoing description of the invention has been presented describing certain operable and preferred embodiments. It is not intended that the invention should be so limited since variations and modifications thereof will be obvious to those skilled in the art, all of which are within the spirit and scope of the invention.

What is claimed is:

1. An adult incontinence kit comprising:
a disposable, re-sealable, impermeable bag;
at least one disposable adult brief;
at least one pair of disposable gloves;
at least one disposable cleansing cloth; and
at least one package of skin care ointment;
wherein the at least one disposable adult brief, at least one pair of disposable gloves, at least one disposable cleansing cloth and at least one package of skin care ointment are housed in the disposable, re-sealable impermeable bag prior to use and wherein the disposable, re-sealable impermeable bag is capable of being used for collecting, sealing and disposing of the at least one disposable adult brief, at least one pair of disposable gloves, at least one disposable cleansing cloth and at least one foil pack of skin care ointment after some or all of the items have been used.

2. The adult incontinence kit as set forth in claim 1, wherein the disposable gloves are latex or non-latex gloves.

3. The adult incontinence kit as set forth in claim 1, wherein the disposable, re-sealable impermeable bag is opaque.

4. The adult incontinence kit as set forth in claim 1, wherein the disposable, re-sealable impermeable bag is a biohazard bag.

5. A method of providing for the incontinency needs of an individual comprising the steps of:
a) providing a disposable, sealed and re-sealable impermeable bag housing at least one disposable adult brief, at least on pair of disposable gloves, at least one disposable cleansing cloth and at least on package of skin care ointment;
b) storing the disposable, sealed and re-sealable impermeable bag in close proximity to an individual suffering from adult incontinence;
c) opening the disposable, sealed and re-sealable impermeable bag and removing the at least one disposable adult brief, at least one pair of disposable gloves, at least one disposable cleansing cloth and at least one package of skin care ointment from the disposable, sealed and re-sealable impermeable bag;
d) putting on the at least one pair of disposable gloves;
e) optionally removing a soiled disposable adult brief from the individual suffering from adult incontinence;
f) using the at least one cleansing cloth to remove soil from the individual suffering from adult incontinence;
g) applying the skin care ointment contained in the package of skin care ointment to the where soil was removed from the individual suffering from adult incontinence;
h) putting the at least one disposable adult brief on the individual suffering from adult incontinence;
i) placing the used pair of disposable gloves, the used cleansing cloth, the used package of skin care ointment and optionally the used disposable adult brief removed from the individual suffering from adult incontinency from step e) in the disposable, re-sealable impermeable bag;
j) resealing the disposable, re-sealable impermeable bag; and
k) transporting the disposable, re-sealable impermeable bag to a disposal receptacle and depositing the disposable, re-sealable impermeable bag therein.
